(19) United States
(12) Patent Application Publication
(10) Pub. No.: US 2014/0200668 A1
(43) Pub. Date: Jul. 17, 2014

(54) IMPLANT SYSTEM HAVING A MULTIFUNCTIONAL INSERTER AND COMPLEMENTARY IMPLANT

(71) Applicant: David Louis Kirschman, Dayton, OH (US)

(72) Inventor: David Louis Kirschman, Dayton, OH (US)

(73) Assignee: X-spine Systems, Inc., Miamisburg, OH (US)

(21) Appl. No.: 13/741,642

(22) Filed: Jan. 15, 2013

(51) Int. Cl. A61B 17/17; A61F 2/44

(52) U.S. Cl. CPC A61B 17/1757; A61F 2/442
USPC 623/17.16; 606/96

(57) ABSTRACT

An implant system having an inserter or implant-drill guide defining a plurality of overlapping bores or elongated passageways. The inserter or implant-drill bit guide provides a drill jig for defining a complementary-shaped drilled-out area in the first and second vertebra. The implant comprises a plurality or lobes or lobate shapes that are adapted in size to be snugly fit into the implant receiving area.

### BACKGROUND OF THE INVENTION

1. Field of the Invention

This invention relates to an implant system and, more particularly, to an implant system having a multi-functional inserter and complementary implant.

2. Background of the Invention

In the past, intervertebral disks that have become degenerated or damaged typically have to be partially or fully removed. Removal of an intervertebral disk can destabilize the spine, making it necessary to replace the intervertebral disk to maintain the height of the spine and/or to fuse the spine. Spinal implants are often used to perform this function.

During a typical spinal implant procedure, an intervertebral disk is removed and one or more implants are inserted in the disk space between neighboring vertebrae.

The disk material between the intervertebral disks was removed and a tube guide with a large foot plate and prongs over an alignment rod and then embedded the prongs into the adjacent vertebrae. The drill guide served to maintain the alignment of the vertebrae and facilitated a reaming out of bone material adjacent to the disk space. The reaming process created a bore to accommodate a bone dowel implant. The drill guide was thereafter removed following the reaming process to allow for the passage of the bone dowel which had an outer diameter significantly larger than the reamed bore and the inner diameter of the drill guide.

In the past, some cages or implants were provided in a cylindrical form and were inserted into complementary-shaped drilled-out areas in the disk area. It was not uncommon that the implants required the use of screws, fasteners and/or plates to retain the cage in its implanted position.

Improvement in the area of spinal implants is needed to reduce the procedure time by utilizing improved instruments and techniques to improve the accuracy with which the implant is implanted in the disk area and to improve the means by which the cage is inserted and retained in the disk area between the adjacent vertebrae.

### SUMMARY OF THE INVENTION

It is, therefore, an object of one embodiment of the invention to provide a system and method for preparing an intervertebral disk area for implantation of an implant.

Another object of one embodiment of the invention is to provide an inserter or insertion instrument that functions as both a drill guide and/or jig and also causes the disk area to be prepared into a predetermined shape suitable for receiving an implant.

Still another object of one embodiment of the invention is to provide an inserter or insertion instrument adapted to receive and guide a drill bit for preparing a disk area into a predetermined shape or configuration such that it has a cross-sectional shape adapted to receive a complementary-shaped implant having a plurality of lobes.

Still another object of one embodiment of the invention is to provide an inserter or insertion instrument having a plurality of intersecting elongated apertures adapted to define an implant receiving area having a predetermined shape suitable for receiving an implant having a complementary shape such that the implant can be guided through the inserter or insertion instrument.

Yet another object of one embodiment of the invention is to provide an inserter or insertion instrument that provides both a drill bit guide and/or drill jig and is also adapted to provide an implant guide for guiding an implant into an implant receiving area prepared by passing a drill bit through the elongated passageways of the inserter or insertion instrument.

Still another object of one embodiment of the invention is to provide a system and procedure for preparing a plurality of seats adapted to receive an implant having an outer wall that is complementarily-shaped.

Still another object of one embodiment of the invention is to provide an implant having at least one or a plurality of lobes and that defines, for example, a bi-lobate shape, a tri-lobate shape or a quad-lobate shape, wherein the axes of the lobes are generally at least one of coplanar, non-coplanar, parallel or non-parallel.

In one aspect, one embodiment of the invention comprises an implant comprising an implant housing having an outer housing wall, the outer housing wall comprising a plurality of implant lobes.

In anther aspect, another embodiment of the invention comprises an implant-drill guide comprising a housing having a plurality of passage wall portions defining a plurality of passages or bores extending through the housing, the plurality of passage wall portions intersecting and cooperating to define at least one implant guide passageway through the housing, the plurality of passages or bores being in communication and each being adapted to receive and guide at least one drill bit for drilling into bone in order to define or provide an implant receiving area for receiving an implant, the at least one implant guide passageway being adapted to receive and guide the implant into the implant receiving area, the implant having a cross-sectional shape that generally complements a cross-sectional shape of the plurality of passages or bores.

In still another aspect, another embodiment of the invention comprises an implant system comprising an implant-drill guide comprising a implant-drill housing having a plurality of passage wall portions defining a plurality of passages or bores extending through the implant-drill housing, the plurality of passages wall portions intersecting and cooperating to define at least one implant guide passageway through the implant-drill housing, the plurality of passages or bores being in communication and at least one of the plurality of passages being adapted to receive and guide at least one drill bit for drilling into bone in order to facilitate defining or providing an implant receiving area for receiving an implant, the implant receiving area having at least one drill lobe area created by the drill bit, the implant having an implant housing having an outer housing wall, the outer housing wall comprising at least one implant lobe that generally complements a shape of and mates with the at least one drill lobe area, the at least one implant guide passageway being adapted to receive and guide the implant into the implant receiving area.

These and other objects and advantages of the invention will be apparent from the following description, the accompanying drawings and the appended claims.

### BRIEF DESCRIPTION OF THE DRAWINGS

FIG. 1 is a perspective view illustrating an introducer or introduction instrument and implant in accordance with one embodiment of the invention;

FIG. 2 is an illustration showing the implant shown in FIG. 1 inserted between a first vertebra and a second vertebra;

FIG. 3 is a view showing the implant situated between the first and second vertebrae from a lateral approach;

FIG. 4 is a generally perspective view of the introducer or introduction instrument shown in FIG. 1;

FIG. 5A is right side view illustrating a housing of the introducer or introduction instrument and illustrating a plurality of male guide protuberances or projections for aligning the introducer or introduction instrument between the first and second vertebrae shown in FIG. 1;

FIG. 5B is a fragmentary view of another embodiment showing a plurality of male guide protuberances or projections having centerlines that are generally offset from a main centerline or longitudinal axis of the introducer or introduction instrument;

FIG. 6 is a plan view of the introducer or introduction instrument shown in FIG. 4 illustrating a window;

FIG. 7 is a end view taken along line 7-7 in FIG. 5A of the introducer or introduction instrument;

FIG. 8 is a view illustrating the insertion of the male guide protuberances or projections between the first and second vertebrae;

FIG. 9 illustrates the introducer or introduction instrument after the male guide protuberances or projections have been inserted between the first and second vertebrae;

FIG. 10 is a view illustrating a drill bit being inserted and guided into the introducer or introduction instrument;

FIG. 11 is a view illustrating the drill bit entering into the disk space between the first and second vertebrae;

FIG. 12A is a view of the introducer or introduction instrument relative to the first and second vertebrae after the male guide protuberances or projections have been inserted between the first and second vertebrae and thereby causing a general alignment of the open-eight shape or double barrel shape of the interior wall of the introducer or introduction instrument relative to the first and second vertebrae;

FIG. 12B is a view similar to FIG. 12A illustrating that the introducer or introduction instrument of FIG. 5A has the male guide protuberances or projections having centerlines that are generally co-linear with the longitudinal axis of the introducer or introduction instrument;

FIG. 13 is a view illustrating the various seats that are created after a drill bit is used to drill elongated passageways, seats or channels in the first and second vertebrae;

FIG. 13A is a view taken along the line 13A-13A in FIG. 13 illustrating the elongated passageways, seats or channels and also illustrating at least one or a plurality of intersecting areas, elongated ridges, projections or rails in accordance with one embodiment of the invention;

FIG. 14A is a view illustrating the implant situated between the first and second vertebrae after the introducer or introduction instrument is used to place the implant therebetween;

FIG. 14B is a view illustrating the implant implanted at a generally lateral approach between the first and second vertebrae;

FIG. 15 is a view of an instrument that can be secured to the implant and used to insert the implant through the introducer or introduction instrument and into an implant area between the first and second vertebrae;

FIG. 16A is an end view of the implant illustrating various features of the implant including the threaded apertures and alignment apertures;

FIG. 16B is a side view of the implant shown in FIG. 16A illustrating a beveled nose of the implant for facilitating inserting the implant through the introducer or introduction instrument and between the first and second vertebrae;

FIG. 16C is a perspective view of the implant in accordance with one embodiment of the invention and illustrating a beveled surface on the front end of the implant;

FIG. 16D is another perspective view illustrating alignment channels and graft receiving areas;

FIG. 17 is an enlarged fragmentary view of a drill bit in accordance with one embodiment of the invention;

FIG. 18 is a perspective view illustrating an introducer or introduction instrument in accordance with the embodiment shown in FIG. 20 and after the introducer or introduction instrument has been positioned for drilling using two different sized drill bits;

FIG. 19 is a perspective view of the implant shown in the embodiment of FIGS. 18-23 and illustrating the different shapes and sizes of implant lobes;

FIG. 20 is a view of an embodiment of an introducer or introduction instrument having a plurality of interior walls adapted not to have the same size or circumference;

FIG. 21 is a view of a drilled-out implant receiving area after use of the introducer or introduction instrument shown in FIG. 20;

FIG. 22 is a view of a multi-lobe shape wherein each lobe has been adapted to have a different size, dimension or circumference and further illustrating the lateral approach in accordance with one embodiment of the invention;

FIG. 23 is a view similar to FIG. 18 except that the disk material has been removed from between the first and second vertebrae and illustrating the implant being inserted into the introducer or introduction instrument;

FIGS. 24A-24D show other illustrative embodiments showing both the introducer or introduction instrument and the associated implant having a plurality of lobes and/or offset lobes; and

FIGS. 25A-25D correspond to the various embodiments of FIGS. 24A-24D, respectively, and illustrate the use of the introducer or introduction instrument of FIGS. 24A-24D and the associated creation of implant-receiving areas and implantation of the implant shown in FIGS. 24A-24D.

### DETAILED DESCRIPTION OF PREFERRED EMBODIMENTS

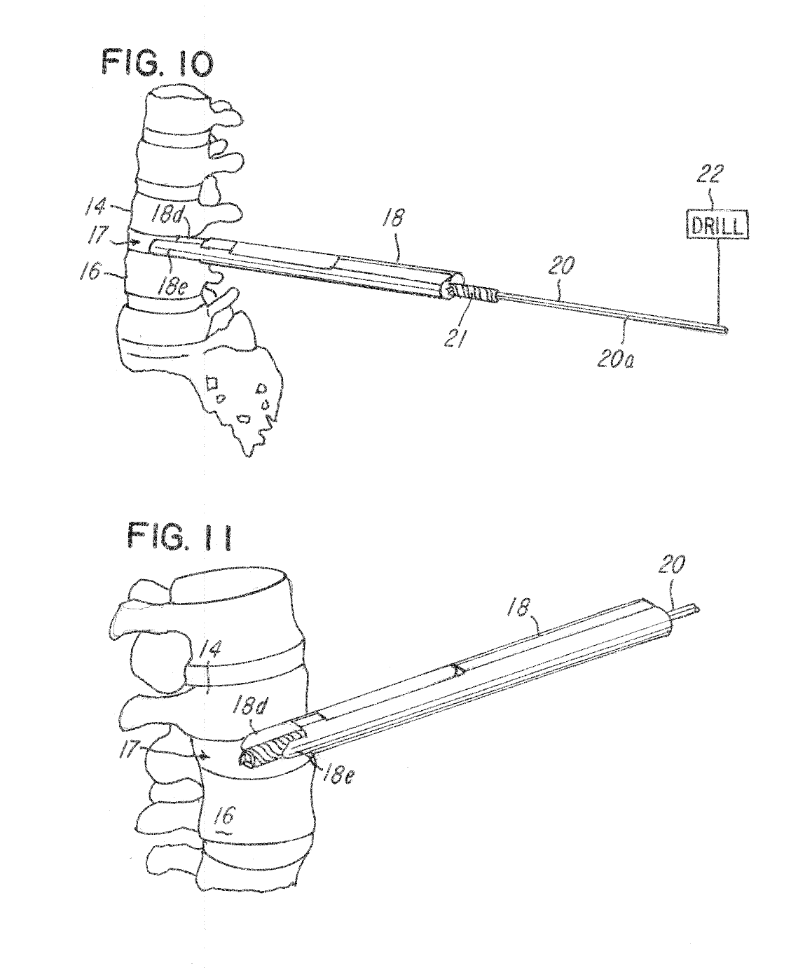

Referring now to FIGS. 1-25D, an implant system 10 is shown. In one embodiment, the implant system 10 is adapted for implanting an implant 12 between a first vertebra 14 and an adjacent second vertebra 16 in the manner described herein. The implant system 10 comprises an introducer or introduction instrument 18 that is adapted to provide a drill guide or drill jig for guiding a drill bit 20 having a shank 20a (FIGS. 10 and 17) toward the first vertebra 14 and second vertebra 16 so that the drill bit 20 may drill a predetermined pattern or aperture, such as a pattern or aperture having a plurality of bores or passageways 24 (FIG. 13) and 26 that overlap as shown. In the illustration being described, the overlapping plurality of bores or passageways 24 and 26 cooperate to define or provide at least one drilled-out implant receiving area 28 for receiving a complementary-shaped implant 12 (FIGS. 14A, 16A and 16D).

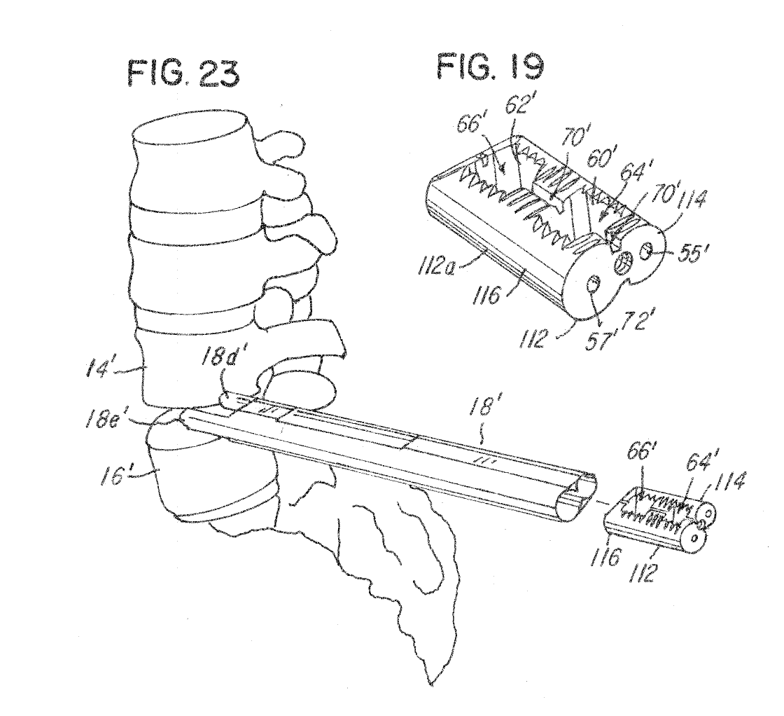

In the illustration being described, note that the introducer or introducer instrument 18 comprises a plurality of wall portions 18a and 18b (FIG. 7) that define a plurality of passageways or bores 30 and 32, respectively, to provide a plurality of pathways or passages through which the drill bit 20 may pass and, eventually, drill into the first vertebra 14 and the second vertebra 16. The plurality of passages or bores 30 and 32 cooperate to define at least one implant guide passageway 31 for receiving the implant 12 and for guiding it to the implant receiving area 28 (FIG. 13). In the example, this drilling provides or defines at least one or a plurality of drill lobe areas or seats 14a (FIG. 13) and 14b in the first vertebra 14 and the least one or a plurality of drill lobe areas or seats 16a and 16b in the second vertebra 16 as best shown in FIGS. 13 and 14A. It should be understood that the at least one or a plurality of drill lobe areas, channels or seats 14a, 14b, 16a, 16b further cooperate to define or provide the implant receiving area 28 (FIG. 28) having a predetermined or predefined cross-sectional shape. As will be described later herein, the implant 12 comprises an exterior wall 12a (FIG. 16D) having a cross-sectional shape that generally complements the cross-sectional shape of the drilled-out implant receiving area 28 (FIG. 19) so that the implant 12 is snugly received in the seats 14a, 14b, 16a and 16b and held in place between the first and second vertebrae 14 and 16 after it is received in the implant receiving area 28, as illustrated in FIG. 14A.

It should be appreciated that the introducer or introduction instrument 18 defines or provides not only a drill jig and guide for guiding at least one or a plurality of drill bits 20, but also a guide for guiding the implant 12 into the implant receiving area 28 after the implant receiving area 28 has been prepared by drilling. Details of the introducer or introduction instrument 18, implant 12 and the implant receiving area 28 will now be described.

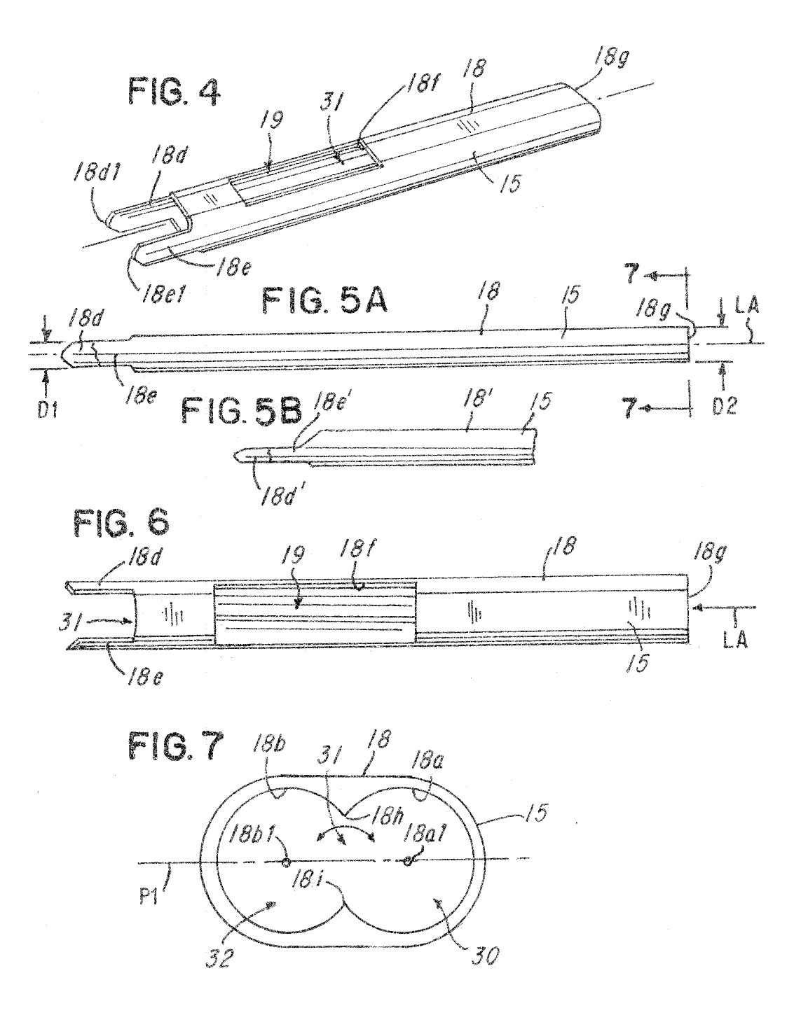

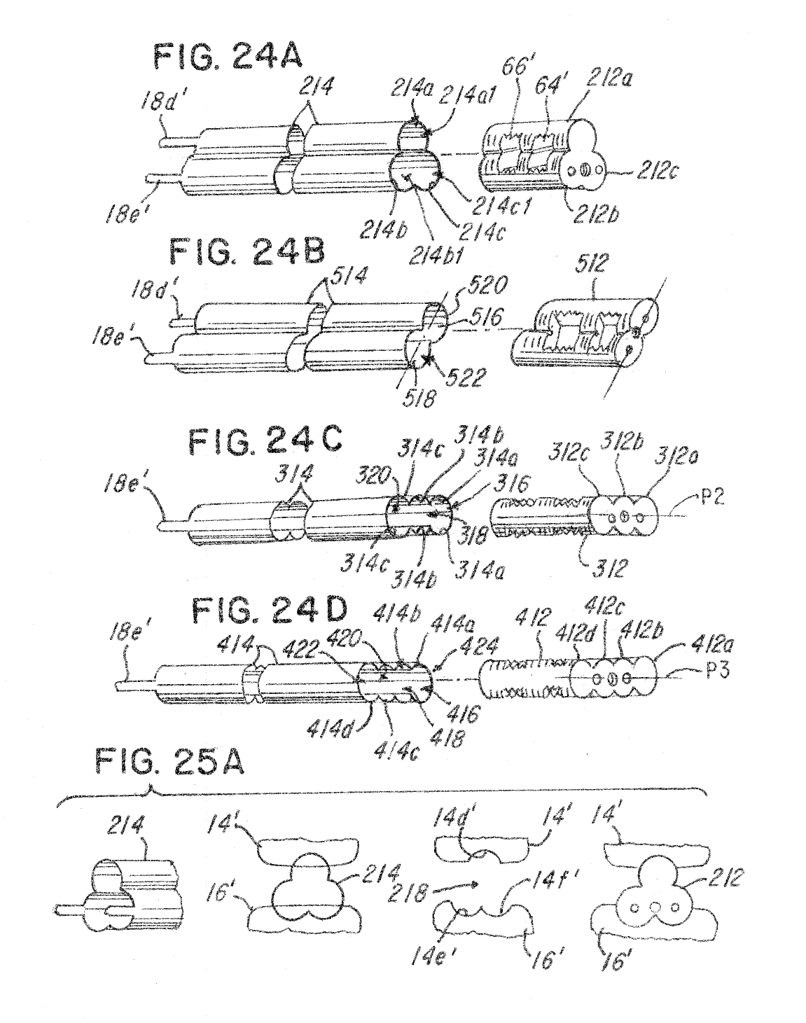

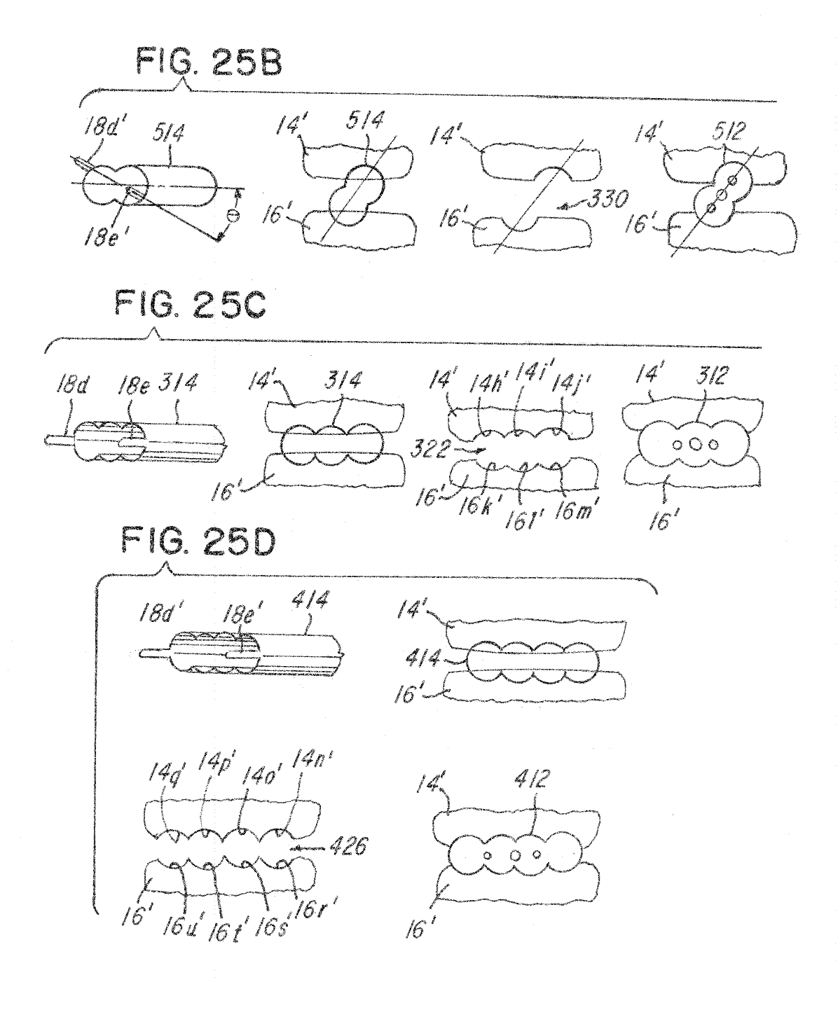

Referring to FIGS. 4-9, note that the introducer or introduction instrument 18 comprises a housing 15 having the plurality of passage wall portions 18a (FIG. 7) and 18b that define or provide the plurality of passages or bores 30 and 32, each adapted to receive and guide the drill bit 20. Note that the plurality of passage wall portions 18a and 18b are generally cylindrical and intersect to define an “open-eight”, double-barrel or bi-lobate shape in the embodiment shown and cooperate to define at least one implant guide passageway 31 (FIG. 7). As will be discussed later herein, the introducer or introduction instrument 18 may comprise a plurality of passage wall portions that define other shapes, such as a tri-lobate (FIGS. 24A, 24C), a quad-lobate shape (FIG. 24D), an offset tri-lobate shape (FIG. 24A) and an offset bi-lobate shape (FIG. 25B), all of which are described later herein.

Referring back to FIG. 7, note that the passage wall portion 18a is generally circumferential and defines a first open circumference or partial circle having a center or axis 18a1, and the passage wall portion 18b defines a second open circumference or partial circle having a center or axis 18b1. Note in the illustration shown in FIG. 7, that the axes 18a1 and 18b1 lie in a common imaginary plane P1 so that the plurality of passages or bores 30 and 32 defined by the passage wall portions 18a and 18b, respectively, are side-by-side and intersecting to define the open-eight, “double barrel” or bi-lobate shape configuration or shape illustrated in FIG. 7. In the illustration being described, the plurality of passages or bores 30 and 32 are generally parallel to a longitudinal axis LA (FIG. 6) extending through the longitudinal length of the introducer or introduction instrument 18. As mentioned earlier, note that each of the plurality of passages or bores 30 and 32 are adapted to receive and guide the drill bit 20. The drill bit 20 has a cross-sectional dimension or diameter that is slightly smaller than a cross-sectional dimension or diameter of the plurality of passages or bores 30 and 32.

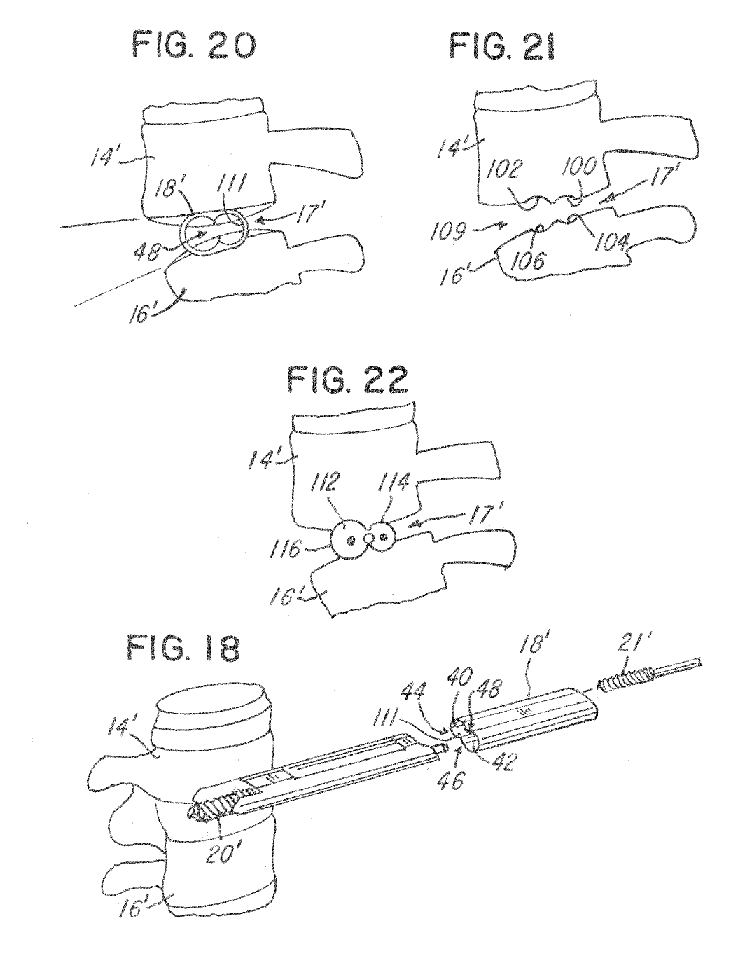

In the illustration shown in FIGS. 1-17, note that the plurality of passages or bores 30 and 32 have substantially the same circumference or partial circumference or circle and are adapted to be substantially the same size, so that they are adapted to receive the same size drill bit 20 which, as mentioned, is slightly smaller. In this illustration, the same drill bit 20 may be used and guided through each of the plurality of passages or bores 30 and 32. As will be described later herein relative to FIGS. 18-23, it should be understood that the introducer or introduction instrument 18 may have a plurality of passage wall portions that define circumferences that are not the same and that are adapted to define a plurality of passageways or bores, such as the plurality of passageways or bores 44 and 46 (FIG. 18) having different circumferences. With such embodiment, a different size drill bit 21′ is used to drill into the first vertebra 14′ and the second vertebra 16′, as illustrated in FIGS. 18 and 21, in order to define an implant receiving area 109 (FIG. 21) having a plurality of different size seats 100, 102, 104 and 106 (FIG. 21) that cooperate to define the implant receiving area 109. In the embodiment of FIGS. 18-23, the passageway or bore 44 (FIG. 18) is larger than the passage or bore 46 so that the passageway or bore 44 can guide a drill bit 20′ (FIG. 18) that is larger than the drill bit 20. This embodiment is adapted to permit the use of a complementary-shaped implant 112 (FIG. 22) having a cross-sectional shape that generally complements the cross-sectional shape of the implant receiving area 109 to enable the implant 112 to be received in the implant receiving area 109. Further details of the embodiment shown in FIGS. 18-23 will be described later herein.

Thus, it should be understood that in the illustrations being described, the plurality of passage wall portions 18a (FIG. 7) and 18b may define at least partial circles or circumferences that are generally the same size or that can be different sizes such that they define through passageways or bores 30, 32, respectively, that are generally the same size or different sizes (illustrated in FIG. 20). This enables the creation of the implant receiving areas 28 (FIG. 13), 109 (FIG. 21) having the predetermined shape that is adapted and created to receive a complementary-shaped implant, such as implants 12 (FIG. 14A) and 112 (FIG. 22) having lobes of the same size or different sizes.

Returning to the embodiments in FIGS. 1-18, note that the introducer or introduction instrument 18 comprises at least one or a plurality of male guide protuberances or projections 18d (FIGS. 1, 4-6) and 18e that are guided into a disk area 17 between the first vertebra 14 and the second vertebra 16. The at least one or a plurality of male guide protuberances or projections 18d and 18e are adapted to provide an aligner or alignment means for aligning the introducer or introduction instrument 18 relative to the disk area 17, the first vertebra 14 and the second vertebra 16 so that the at least one drill bit 20 becomes aligned as desired and can drill a desired pattern of elongated bores, seats or channels 14a, 14b, 16a and 16b into at least one of the first vertebra 14 or second vertebra 16 when the drill bit 20 is guided through the introducer or introduction instrument 18 and drills the first and second vertebrae 14 and 16. The at least one or plurality of male guide protuberances or projections 18d and 18e have angled or beveled ends 18d1 (FIG. 4) and 18e1, respectively, to facilitate insertion between the first and second vertebrae 14 and 16.

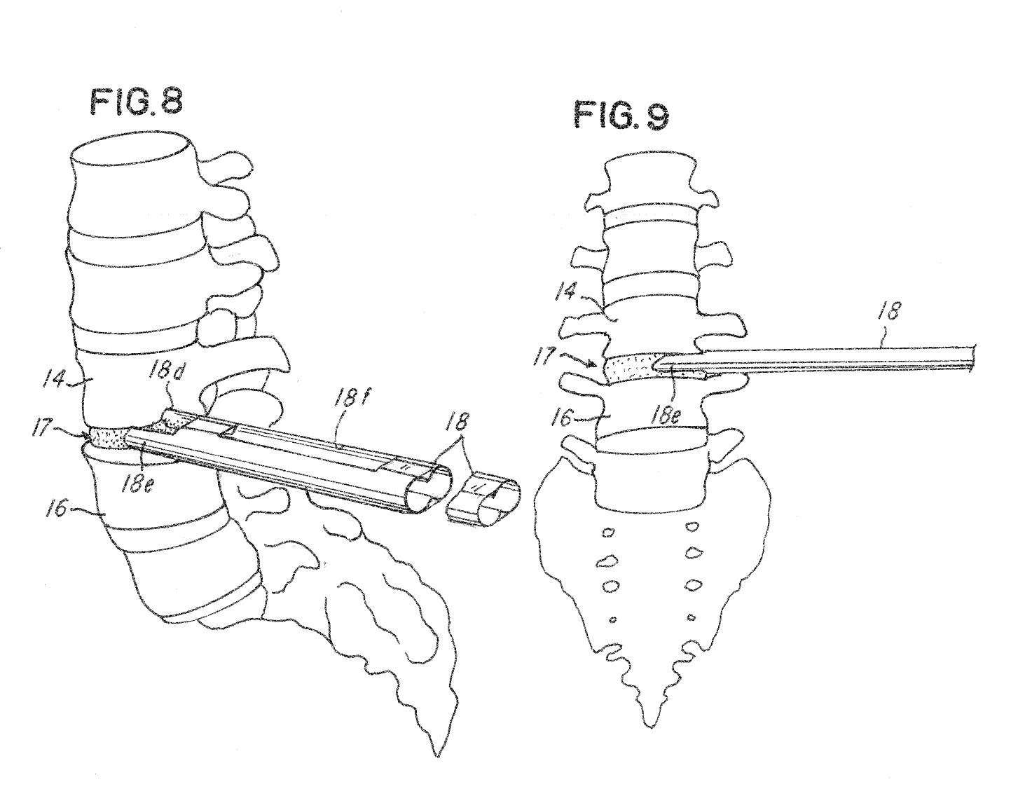

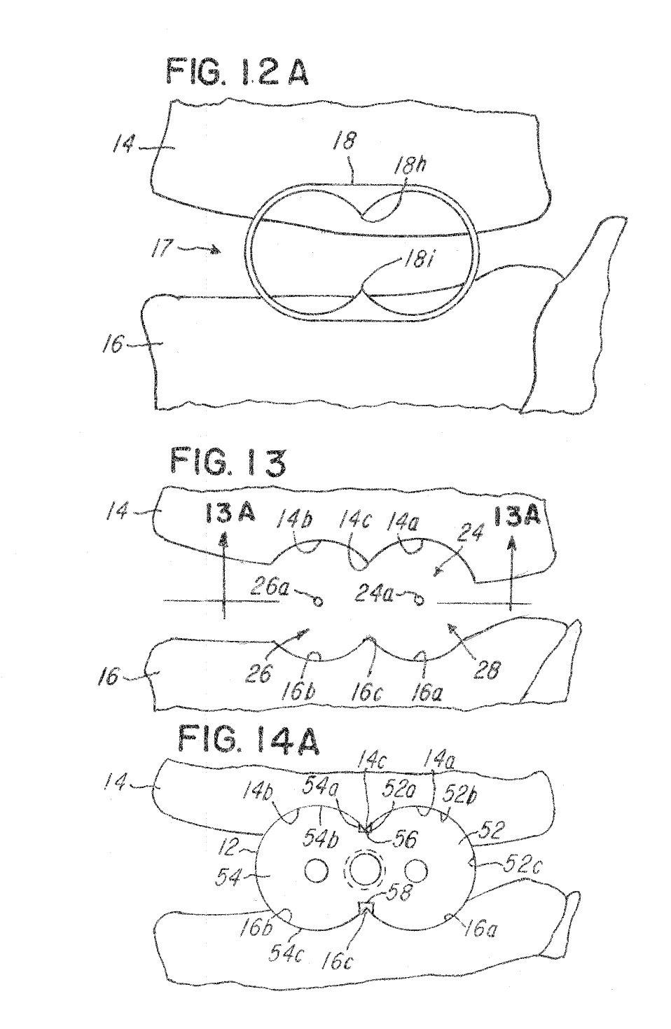

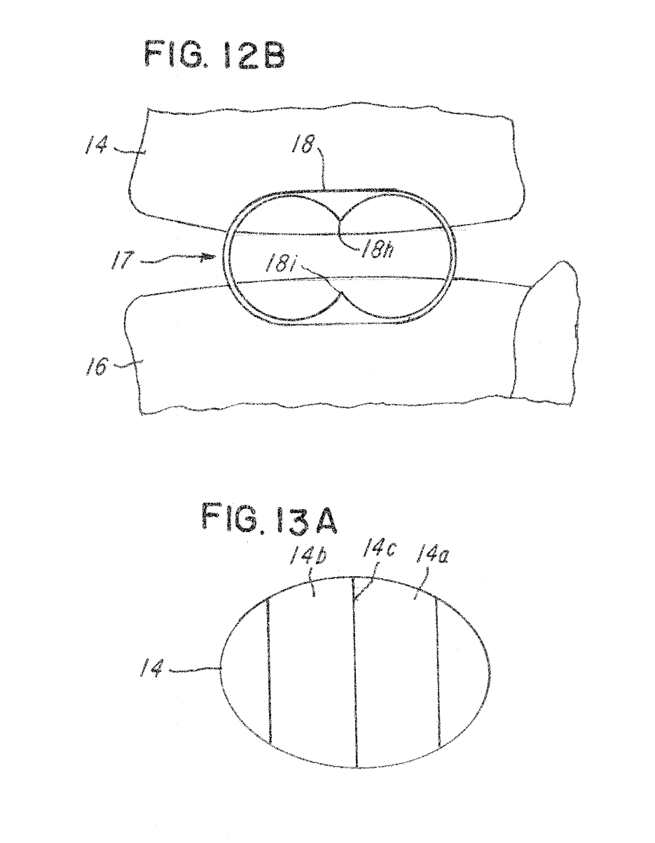

As illustrated in FIGS. 8 and 9, it should be understood that the plurality of male guide protuberances or projections 18d and 18e provide the aligner or alignment means for aligning the introducer or introduction instrument 18 into a predetermined position relative to the first and second vertebrae 14 and 16 so that the desired shape or pattern may be drilled. Thus, the introducer or introduction instrument 18 provides a drill jig that causes the plurality of passages or bores 30 and 32 to become aligned with the first and second vertebrae 14 and 16 as illustrated in FIGS. 12A and 12B when the male guide protuberances or projections 18d and 18e are inserted between the first and second vertebrae 14 and 16. This causes the drill bit 20 to become aligned to drill the plurality of drill lobe areas or seats 14a, 14b, 16a and 16b.

Note that the plurality of male guide protuberances or projections 18d and 18e have a height or dimension D1 (FIG. 5A) that is smaller than the height or dimension D2 of the introducer or introduction instrument 18 as shown. The male guide protuberances or projections 18d and 18e each have a longitudinal axis that lies in the same plane as the longitudinal axis LA of the introducer or introduction instrument 18 and each are generally symmetrical about their longitudinal axis, and the longitudinal axis LA which causes the plurality of passages or bores 30, 32 to be aligned substantially equally or midway between with the first and second vertebrae 14 and 16 as shown in FIG. 12B. However, it should be understood that they could be asymmetrical about the axis LA or about their own longitudinal axis in order to adapt to or accommodate a local anatomy. For example, in another embodiment, male guide protuberances or projections 18d′ and 18e′ are arranged as illustrated in FIG. 5B, thereby causing a greater portion of the plurality of passages or bores 30 and 32 to become aligned with the first vertebra 14 and a smaller portion with the second vertebra 16, as illustrated in FIG. 12A. This enables the drilling and creation of drill lobe areas or seats 14a, 14b (FIG. 13), for example, to be created in the first vertebra 14 that are relatively larger or “deeper” than the drill lobe areas or seats 16a and 16b that are created, reamed or drilled in the second vertebra 16. Thus, the offset male projections 18d′ and 18e′ (FIG. 5B) enable and cause a smaller portion or cross-sectional area of the passageways or bores 30 and 32 to overlap and align with the second vertebra 16 resulting in less bone being removed by the drill bit 20, thereby making the seats 16a and 16b generally smaller in cross-section by comparison to the seats 14a, 14b in FIG. 12A.

As mentioned and illustrated, the dimension D1 (FIG. 5A) is generally smaller than a cross-sectional height or dimension D2 of the introducer or introduction instrument 18, thereby permitting the plurality of male guide protuberances or projections 18d and 18e to be received in the implant receiving area 28 between the first and second vertebrae 14 and 16 so that the plurality of passages or bores 30 and 32 may become aligned with vertebrae 14 and 16 so that the drill bit 20 can drill or ream out and create implant receiving area 28 (FIG. 13) between the first and second vertebrae 14 and 16 and also to be used to guide the implant 12 thereto as illustrated in FIGS. 1-3.

Advantageously, the introducer or introduction instrument 18 is adapted to define both a drill jig for guiding at least one drill bit 20 through each of the plurality of passages or bores 30 and 32 and a guide and alignment tool for guiding and aligning the implant 12 toward and into the implant receiving area 28.

Returning to FIGS. 1-7, a first one of the plurality of passages or bores 30 defines a first generally elongated passageway through the introducer or introduction instrument 18 housing 15, and the second one of the plurality of passages or bores 32 defines a second generally elongated passageway through the introducer or introduction instrument 18 housing 15. As mentioned earlier herein, it is important to note that the first and second generally elongate passageways or bores 30, 32 are in communication and intersect radially and are at least partly cylindrical or circular when viewed in cross-section in one illustrative embodiment. Note, however, that because of the intersection of the plurality of passages or bores 30 and 32, they do not define complete circles, but rather define the “open-eight”, multi-lobe or double barrel shape mentioned earlier herein. Again, the first and second plurality of passages or bores 30, 32 may be generally the same cross-sectional size or may be different sizes or shapes in cross-section. Thus, it is important to note that the plurality of passage wall portions 18a and 18b cooperate to define a multi-lobe passageway shape, which in this embodiment is a dual-lobe shape. As mentioned earlier herein, the drill bit 20 is received in and guided through the first and second elongated passageways or bores 30, 32 toward the first and second vertebrae 14 and 16 so that a drill 22 (FIG. 10) with the drill bit 20 can drill out at least a portion of the first vertebra 14 and second vertebra 16, as best illustrated in FIGS. 10-13, thereby creating the plurality of drill lobe areas or seats 14a, 14b, and 16a, 16b in the first and second vertebrae 14 and 16, respectively, as well as the implant receiving area 28 (FIG. 13). The plurality of drill lobe areas or seats 14a, 14b, 16a and 16b define the plurality of arcuate or curved seats that are adapted to receive the lobes, such as lobes 52, 54 of the generally complementary-shaped implant 12 as shown in FIG. 14A.

As illustrated in FIGS. 4, 6 and 8, notice that the introducer or introduction instrument 18 housing 15 comprises a generally rectangular internal wall or edge 18f that defines or provides at least one window 19 (FIGS. 4 and 6) for viewing the implant guide passageway 31 inside the introducer or introduction instrument 18. In the illustration being described, the at least one window 19 is adapted for viewing the implant 12 and/or drill bit 20 as they pass through at least one or both of the plurality of passages or bores 30 or 32.

Referring now to FIGS. 14A-16B, details of the implant 12 will now be described. The implant 12 comprises outer housing wall 12a. The outer housing wall 12a comprises the first and second generally cylindrical lobes 52 and 54. Note that in the illustration shown in FIGS. 14A-16B, the first and second generally cylindrical lobes 52 and 54 are generally the same size, but as mentioned earlier herein, the plurality of lobes 52 and 54 could define different sizes or shapes that generally match or complement the size or shape of the plurality of passages or bores, such as bores 30, 32 (FIG. 7) and 40, 42 (FIG. 18).

The implant 12 may comprise at least one or a plurality of interior walls 60 and 62 (FIG. 16D) that define a plurality of interior graft receiving areas 64 and 66 for receiving graft material. In the illustration being described, the implant 12 is loaded or packed with graft material prior to insertion into the introducer or introduction instrument 18. The implant 12 may also comprise at least one or a plurality of teeth or serrations 68 which are adapted to facilitate retaining the implant 12 in the drill lobe areas or seats 14a, 14b, 16a and 16b and in a fixed position between the first vertebra 14 and the second vertebra 16.

It should be understood that the exterior wall 12a and the first and second generally cylindrical lobes 52 and 54 of the implant 12 cooperate to define a bi-lobate, double barrel or an “open-eight” shape in cross-section that generally complements the “open-eight”, double barrel or bi-lobate shape defined by the interior wall portions 18a, 18b and implant guide passageway 31 (FIG. 7) of the introducer or introduction instrument 18, but is slightly smaller so that the implant 12 can be easily guided and passed through the implant receiving area 31 of the introducer or introduction instrument 18 and into the implant receiving area 28 (FIG. 13) after it has been drilled out and created.

In the illustration being described, an end 12a1 (FIGS. 16B, 16C and 16D) of the implant 12 comprises a beveled surface 12a2 (FIG. 16C) that facilitates guiding the implant 12 into both the introducer or introduction instrument 18 and the implant receiving area 28.

Another feature of one embodiment of the invention being described is that it has an aligner or alignment means (FIGS. 13, 13A, 14A and 16A and 16B) for aligning and guiding the implant 12 into and through the introducer or introduction instrument 18 and into the implant receiving area 28. The implant 12 comprises a first joining wall 56 (FIGS. 14A and 16B) and a generally opposing second joining wall 58 which joins the first and second generally cylindrical lobes 52 and 54 as illustrated in FIG. 16A. In this regard, the first joining wall 56 is coupled to and joins a first portion 52a of the first generally cylindrical lobe 52 with a first portion 54a of the second generally cylindrical lobe 54. The first joining wall 56 cooperates with those first portions 52a and 54a to define a first generally U-shaped channel 70 (FIG. 16A). Likewise, the second joining wall 58 joins a second portion 54b of the first generally cylindrical lobe 52 with a second portion 54b of the second generally cylindrical lobe 54 to define a second generally U-shaped second channel 72 (FIG. 16A).

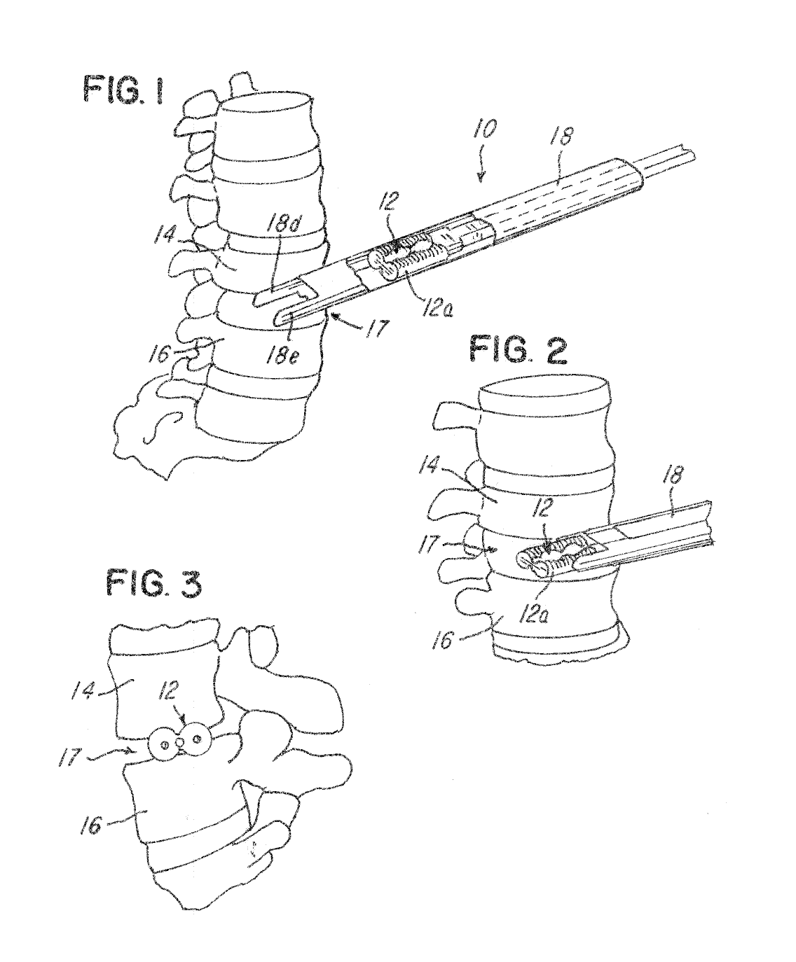

Notice that the introducer or introduction instrument 18 comprises projections, rails, ridges or guides 18h, 18i (FIGS. 7, 12A and 13A) at the intersection between the first wall portions 18a and 18b. These projections, rails ridges or guides 18h, 18i are received in the channels 70, 72, respectively, and facilitate aligning and guiding the implant 12 through the introducer or introduction instrument 18 and toward and into the implant receiving area 28 (FIGS. 1 and 2).

As best illustrated in FIGS. 13 and 13A, notice that after the drill bit 20 is passed through each of the bores 32 and 34 and drills out the first and second vertebrae 14 and 16, as illustrated in FIG. 13, the at least one or a plurality of intersecting areas, elongated ridges, projections or rails 14c and 16c are defined or created at the intersection of the seats 14a, 14b and 16a, 16b, respectively. The intersecting areas, elongated ridges, projections or rails 14c and 16c are generally elongated and extend into or across the first vertebra 14 and second vertebra 16 in the implant receiving area 28. For example, the plurality of intersecting area, elongated ridge, projection or rail 14c extends along across the first vertebra 14, as illustrated in FIG. 13A. Likewise, the intersecting area, elongated ridge, projection or rail 16c similarly extends across the second vertebra 16.

It should be understood that an advantageous feature of the embodiment being described is that the aligners or alignment means defined by the at least one or a plurality of the intersecting areas, elongated ridges, projections or rails 14c and 16c align with the projections, rails, ridges or guides 18h, 18i, respectively, when the introducer or introduction instrument 18 is positioned adjacent the first and second vertebrae 14, 16 after the plurality of bores or passageways 24 and 26 have been drilled or bored. The plurality of intersecting areas, elongated ridges, projections or rails 14c and 16c are received in the elongated slots or channels 70 and 72, respectively, of the implant 12 and facilitate guiding and aligning the implant 12 in a desired position between the first vertebra 14 and second vertebra 16, as illustrated in FIGS. 1-3 and 14A. Advantageously, this feature also facilitates causing a first wall portion 52b (FIG. 14A) of the first generally cylindrical lobe 52 of implant 12 and a second wall portion 52c of the first generally cylindrical lobe 52 to become operatively aligned with and positioned in the drill lobe areas or seats 14a and 16a, respectively, of the first vertebra 14 and second vertebra 16. Likewise, the second generally cylindrical lobe 54 comprises the first wall portion 54b and second wall portion 54c that become operatively positioned and seated in the drill lobe areas or seats 14b and 16b, respectively.

As mentioned earlier, the implant 12 defines the “open-eight”, double barrel or bi-lobate shape in cross-section similar to the cross-sectional shape defined by the joining passage wall portions 18a and 18b (FIG. 7) of the introducer or introduction instrument 18. Each of the generally cylindrical lobes 52 (FIG. 16) and 54 of the implant 12 have a longitudinal axis A1 and A2, respectively, that become co-axial with the axes 18a1 (FIG. 7) and 18b1 when the implant 12 is inserted into the introducer or introduction instrument 18. Note also that the drilled-out plurality of bores or passageways 24 and 26 generally comprise axes 24a (FIG. 13) and 26a and the axes A1 and A2 of the first and second generally cylindrical lobes 52 and 54 become generally co-axial with these center lines or axes 24a and 26a of the drilled-out passageways 24 and 26, respectively, when the implant 12 is inserted into the implant receiving area 28.

The general procedure and use of the implant system 10 will now be described relative to FIGS. 1-17. First, the introducer or introduction instrument 18 is positioned as illustrated in FIGS. 1 and 8-9 by inserting the male guide protuberances or projections 18d and 18e in the disk area 17 between the first vertebra 14 and second vertebra 16. Advantageously, the introducer or introduction instrument 18 may be positioned for a lateral approach, as illustrated in FIG. 9, or from another approach, such as an anterior approach. Once the introducer or introduction instrument 18 is positioned as illustrated, it provides or defines the drill jig for drilling the predetermined shape or pattern that defines the implant receiving area 28 (FIG. 13) in the first and second vertebrae 14 and 16. The user inserts the drill bit 20 into the bore 30 and the introducer or introduction instrument 18 enables or causes it to be guided along the longitudinal axis 18a1 of the bore 30 as shown in FIGS. 10 and 11. The drill bit 20 is conventionally coupled to the drill 22 (FIG. 10) which the user uses to drill into at least one or both of the first and second vertebrae 14 and 16 as illustrated in FIGS. 12A, 12B and 13. It should be understood that the drill bit 20 drills into the opposing first and second vertebrae 14 and 16 substantially equally in the illustration. However, in the example in FIG. 12A, which illustrates a lateral approach with the introducer or introduction instrument 18 of the embodiment in FIG. 5B, more of the first vertebra 14 is drilled compared to the second vertebra 16.

After the user inserts and drills out the bore or passageway 24 (FIG. 13) to create the first seats 14a and 16a, the drill bit 20 is removed and inserted through the second bore 32 (FIG. 7) and the bore or passageway 26 (FIG. 13) is drilled out and created, thereby creating the second seat 14b in the first vertebra 14 and the generally opposing second seat 16b in the second vertebra 16, as illustrated in FIG. 13. It should be understood that a normal tension and resistance facilitates preventing the first and second vertebrae 14 and 16 from separating during the drilling process. Also, the male guide protuberances or projections 18d and 18e of the introducer or introduction instrument 18 are adapted and sized to fit and impact tightly between the first and second vertebrae 14 and 16 so that there is tension on the disk annulus and the first and second vertebrae 14 and 16, which further facilitates preventing the first and second vertebrae 14 and 16 from separating during drilling. The male guide protuberances or projections 13d and 18e could be the same size or dimension or could be different. For example, they could have different lengths, widths and heights.

As mentioned earlier relative to FIG. 5B, the male guide protuberances or projections 18d and 18e may comprise different widths or different dimensions D1, D2 (FIG. 5A) and may be adapted and sized to fit in the disk space 17, particularly when a lateral insertion approach is taken. Thus, for example, in the illustration shown in FIGS. 1 and 2, it may be desired to use an introducer or introduction instrument 18 having the first male protuberance or projection 18d having dimension D1 (FIG. 5A) that is smaller than dimension D2 of the second male protuberance or projection 18e as the disk area 17 (viewed from right or posterior to left or anterior in the FIG. 12A) gets larger. The male protuberances or projections 18d and 18e, although having different dimensions D1 and D2 would be aligned, similar to the embodiment illustrated in FIG. 5A, to have a centerline axis that is aligned with the centerline axis of the housing 15. Note that when taking an anterior approach, as illustrated in FIG. 12B, the drill bit 20 drills into the opposing vertebrae 14 or 16 substantially equally as illustrated in FIG. 12B. This is to be contrasted with the embodiment shown and described relative to FIG. 5B wherein the centerline axis of the first and second male protuberances or projections 18d and 18e are vertically offset from the centerline or longitudinal axis off the introducer or introduction instrument 18.

After the plurality of bores or passageways 24 and 26 (FIG. 13) and the corresponding drill lobe areas or seats 14a, 16a and 14b, 16b, respectively, are drilled (FIGS. 10 and 11) into the first and second vertebrae 14 and 16, respectively, by passing the drill bit 20 through the passages or bores 30, 32, the drill bit 20 and its shank 20a (FIG. 10) are removed from the bore 32. As mentioned earlier, the overlapping plurality of bores or passageways 24 and 26 and the space or area 17a (FIG. 13) therebetween defines the implant receiving area 28.

The graft areas 64 and 66 of the implant 12 are loaded with graft material (not shown) prior to insertion. In the example, the generally U-shaped channels 70 and 72 are not loaded with graft material so that they can freely receive the intersecting areas, elongated ridges, projections or rails 14c and 16c, respectively.

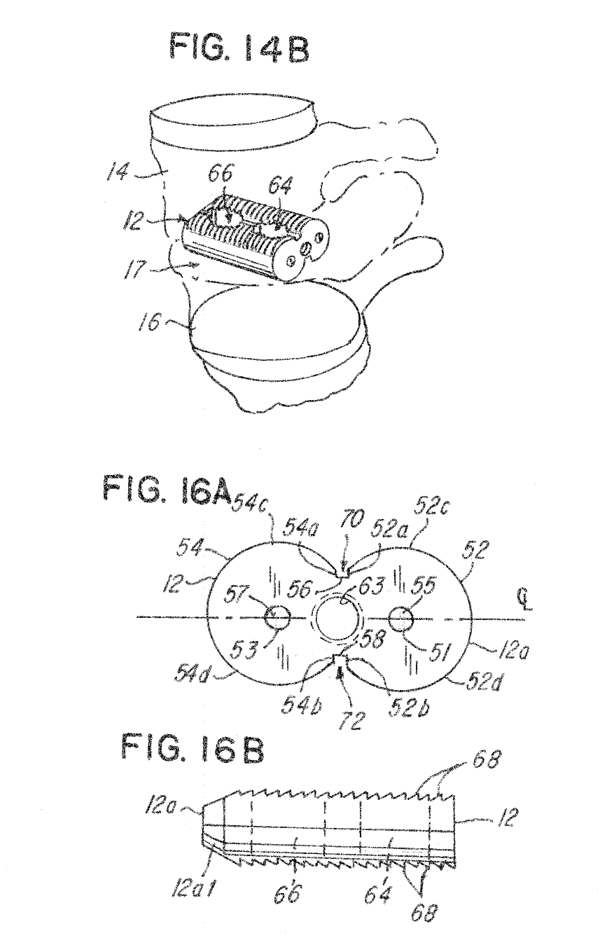

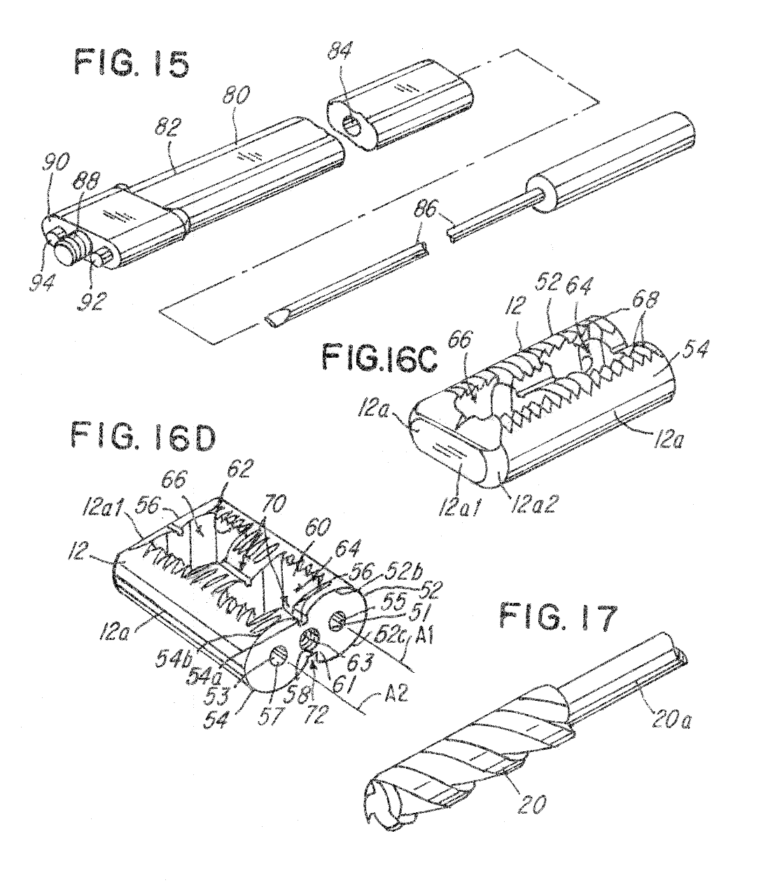

The introducer or introduction instrument 18 remains in place after drilling and the user now inserts the loaded implant 12 into the at least one implant guide passageway 31 (FIG. 7) of the introducer or introduction instrument 18. To facilitate such insertion, the implant system 10 may comprise a tool or inserter 80 (FIG. 15) having a first member 82 and an aperture 84 for receiving a rotatable tool 86 having a male threaded end 88 as shown. The tool or inserter 80 comprises an end 90 through which the male threaded end 88 (FIG. 15) rotatably extends and may also comprise a plurality of guide or locating pins 92 and 94 that are received in apertures 55 (FIG. 16A) and 57, respectively, which are defined by interior generally cylindrical walls 51 and 53 as illustrated in FIG. 16B. The implant 12 further comprises a threaded wall 63 (FIG. 16A) defining a threaded aperture for threadably receiving the male threaded end 88 (FIG. 15) for securing the implant 12 to the tool or inserter 80. First, the user mounts the implant 12 onto the tool or inserter 80 and then may load the graft receiving areas 64 and 66 as mentioned earlier in a manner conventionally known.

The user then uses the tool or inserter 80 to insert the implant 12 into the end 18g (FIG. 4) of the introducer or introduction instrument 18 and guides the implant 12 toward the implant receiving area 28 (FIG. 13). Note that the at least one window 19, which is defined by the internal wall or edge 18f mentioned earlier, provides the user with visual feedback as the implant 12 approaches the implant receiving area 28. In this regard and as mentioned earlier herein, the end 12a1 of the implant 12 may comprise the beveled surface 12a2 for facilitating guiding the implant 12 into the implant receiving area 28.

The user continues inserting the implant 12 through the introducer or introduction instrument 18 and into the implant receiving area 28 until it is securely positioned between the first vertebra 14 and second vertebra 16 as illustrated in FIGS. 2, 14A and 14B. Again, it should be understood that the implant lobes 52 and 54 are adapted and sized to be received in the drill lobe areas or seats 14a, 16a, 14b and 16b, respectively, so that the first and second vertebrae 14 and 16 apply a natural or normal tension thereto, thereby keeping the implant 12 secured between the first and second vertebrae 14 and 16. The plurality of teeth or serrations 68 also facilitate preventing withdrawal of the implant 12 after it is positioned between the first vertebra 14 and second vertebra 16. Although not shown, the implant 12 may have apertures (not shown) for receiving at least one or a plurality of bone screws (not shown) for screwing and securing the implant 12 into the first and second vertebrae 14 and 16 after the implant 12 is positioned. In a preferred embodiment, the plurality of bone screws are not necessary because a natural tension of the implant 12 and wall portions 52c, 54c and 52d, 54d in the drill lobe areas or seats 14a, 16a and 14b, 16b, respectively, and the teeth or serrations 68 hold the implant 12 in place in the implant receiving area 28.

As mentioned earlier herein, one advantageous feature of the illustration being described, is that the introducer or introduction instrument 18 comprises the overlapping generally cylindrical bores 30 and 32 which, after the drill bit 20 has drilled into the first and second vertebrae 14 and 16, define or provide the intersecting areas, elongated ridges, projections or rails 14c and 16c (FIGS. 13 and 14A) that provide or define the aligner or alignment means described earlier. As mentioned previously, the projections, rails ridges or guides 18h, 18i of introducer or introduction instrument 18 and the intersecting areas, elongated ridges, projections or rails 14c and 16c are received in the guide slots or elongated channels 70 (FIG. 16A) and 72, respectively, and facilitate guiding the implant 12 into a predetermined or desired position in the implant receiving area 28 (FIG. 13) between the first vertebra 14 and second vertebra

Referring now to FIGS. 18-24D, other embodiments of the invention are shown. Those parts that are the same or similar to the parts shown in the embodiment described earlier relative to FIGS. 1-16B have the same part numbers except that a “′” (prime mark) has been added to the part numbers in FIGS. 18-24D. As illustrated in FIG. 18, the introducer or introduction instrument 18′ has an interior wall 111 (FIGS. 18 and 20) defined by a first interior wall portion 40 and a second interior wall portion 42 that define a first elongated passageway or bore 44 and a second elongated passageway or bore 46, respectively. The first and second elongated passageways or bores 44 and 46 have different sizes and/or circumferences in cross-section and are adapted to receive different sized drill bits 20′ and 21. Note, for example, in FIG. 18 that the drill bit 21 (FIG. 18) has a smaller diameter than the drill bit 20′. This results in an introducer or introduction instrument 18′ having an internal bi-lobate shape that has two apertures or bores that are not of the same size. It has been found that this shape is particularly suited for use in a lateral insertion approach, as illustrated in FIGS. 20-23, because the disk area 17′ tends to increase in size, as illustrated in FIG. 21, from the posterior portion of the disk area 17′ toward the anterior portion of the disk area 17′ (i.e., when viewed right to left in FIG. 21).

The method and procedure for creating the implant receiving area 109 (FIG. 21) is substantially the same as described earlier herein relative to the embodiment shown and described in FIGS. 1-18. As with the embodiment described earlier herein, the user drills the first and second vertebrae 14′ and 16′ to create the implant receiving area 109, as illustrated in FIG. 21, using the drill bits 20′ (FIG. 18) and 21. After drilling the first vertebra 14′ and the second vertebra 16′ using the different sized drill bits 20′ and 21 guided and inserted through the introducer or introduction instrument 18′, the seats 100 and 102 are created in the first vertebra 14′ and the generally opposing seats 104, 106 are created in the second vertebra 16′ to create or provide at least one implant receiving area 109. A complementary-shaped implant 112 (FIGS. 19, 22 and 23) is inserted and passed through the implant receiving area 48 (FIG. 18) of the introducer or introduction instrument 18′ using the tool or inserter 80 (FIG. 15) until it becomes positioned in the at least one implant receiving area 109 between the first vertebra 14′ and the second vertebra 16′ so that lobe 114 becomes seated in and between seats 100 and 104 and lobe 116 becomes situated in and between seats 102 and 106. The plurality of seats 100, 102, 104 and 106 are adapted to receive the implant 112 (FIGS. 22 and 23) and lobes 114 and 116 that are of different circumferences in cross-section. As with the first embodiment, the circumferences of lobes 114 and 116 are generally the shape and size of their associated seats 100-106. The implant 112 (FIG. 19) also has the external wall 112a that defines the unequal bi-lobate shape that generally complements, but is slightly smaller than, the bi-lobate shape defined by the interior wall 111 (FIGS. 18 and 20) so that the implant 112 can easily pass through the introducer or introduction instrument 18′.

As mentioned earlier herein, the embodiments shown in FIGS. 1-17 illustrate a bi-lobate shape wherein the lobes, such as lobes 52 and 54, have generally the same size or circumference. In contrast, the embodiment illustrated in FIGS. 18-23 shows a bi-lobate shape having lobes 114, 116 that are not the same size or circumference when viewed in cross-section. Of course, it should be understood that other configurations of both the introducer or introduction instrument 18′ and associated implant may be provided.

As mentioned earlier, the introducer or introduction instrument 18 and implant 112 may have more than two lobes. FIGS. 24A and 25A illustrates a tri-lobate implant 212 and introducer or introduction instrument 214. Note that each of the internal walls 214a, 214b and 214c of the introducer or introduction instrument 214 each have a substantially the same shape or circumferential dimension when viewed in cross-section. The internal walls 214a, 214b and 214c define a plurality of passageways or bores 214a1, 214b1 and 214c1, respectively, that overlap as illustrated. The implant 212 also has complementary-shaped lobes 212a, 212b and 212c as shown. During insertion, the introducer or introduction instrument 214 is positioned between the first and second vertebrae 14 and 16 and the drill bit 20 is passed through the plurality of passageways or bores 214a1, 214b1 and 214c1 to drill the first and second vertebrae 14 and 16 to provide the tri-lobate implant receiving area 218 (FIG. 25A) and seats 14d′, 14e′ and 14f′. The introducer or introduction instrument 214 and tool or inserter 80 (FIG. 5) is then used to insert the implant 212 in the tri-lobate implant receiving area 218. In this embodiment, the lobes 212a, 212b and 212c do not lie in the same plane, but have axes that are generally parallel and cooperate to form an imaginary triangle when the implant 212 is viewed from an end.

FIGS. 24C, 25C, 24D and 25D each illustrate tri-lobate and quad-lobate embodiments, respectively. FIG. 24C shows an introducer or introduction instrument 314 having a plurality of wall portions 314a, 314b and 314c that define a plurality of passages 316, 318 and 320, respectively, that at least partially overlap and that comprise substantially the same shape or circumferential dimension in cross-section. Note that the corresponding implant 312 in this embodiment also comprises a complementary tri-lobate shape having lobes 312a, 312b and 312c each having substantially the same circumferential dimension when viewed in cross-section. As shown in FIG. 25C, the introducer or introduction instrument 314 is used with drill bit 20 to drill and provide the implant receiving area 322 and drilled seats 14h′, 14i′, 14y and the generally opposing seats 16k′, 16l′, 16m′, respectively. The implant 312 is adapted to have a complementary tri-lobate shape and sized to conveniently pass through an implant receiving area 322 defined by the overlapping bores 316, 318 and 320 of the introducer or introduction instrument 314 and into the drilled-out implant receiving area 322. Note that the central axes of the lobes 312a, 312b and 312c all lie in a common imaginary plane P2 (FIG. 24C).

FIGS. 24D and 25D illustrate another embodiment. In this embodiment, a four lobe or quad-lobate introducer or introduction instrument 414 and implant 412 is provided. The introducer or introduction instrument 414 comprises a plurality of wall portions 414a, 414b, 414c and 414d that define a plurality of elongated passageways 416, 418, 420 and 422, respectively, as shown and that cooperate to define an implant receiving area 424 for receiving an implant 412.

Note that the implant 412 comprises a similar complementary-shaped configuration having four lobes 412a, 412b, 412c and 412d that are adapted and sized to be received in and to be able to pass through the passageways 416, 418, 420 and 422, respectively, and into the implant receiving area 424 of the introducer or introduction instrument 414 and into a complementary-shaped drilled-out implant receiving area 426 defined by seats 14n′, 14o′, 14p′, 14q′, 16r′, 16s′, 16t′ and 16u′ and the area between the first and second vertebrae 14 and 16.

Like the embodiment of FIG. 24C, the four lobe or quad-lobate introducer or introduction instrument 414 in the embodiment in FIG. 24D have axes that lie in a generally common imaginary plane P3.

In the embodiment illustrated in FIGS. 24B and 25B, an introducer or introduction instrument 514 is shown having walls 516 and 518 that define a plurality of passages or bores 520, 522, respectively. The center lines or axes of the plurality of passageways or bores 520 and 522 of the introducer or introduction instrument 514 lie in a plane that forms a predetermined angle Θ with respect to the longitudinal axis of the plurality of male guide protuberances or projections 18d′ and 18e′ of the introducer or introduction instrument 514 so that when the plurality of male guide protuberances or projections 18d′ and 18e′ are positioned between the first and second vertebrae 14′ and 16′, the plurality of passageways or bores 520 and 522 become offset or angled at the predetermined angle Θ relative to the spinal axis or an imaginary plane defined by an implant receiving area 530 as shown in FIG. 25B. The introducer or introduction instrument 514 comprises the plurality of male guide protuberances or projections 18d′ and 18e′ that lie in an imaginary plane that is offset, angled or tilted with respect to the plane in which the male guide protuberances or projections 18d′ and 18e′ lie. The angular offset creates the implant receiving area 530 to be drilled into the first and second vertebra 14′ and 16′. When the implant 512 becomes situated in the implant receiving area 530, the implant 12 becomes situated at a predetermined angle relative to an axis of the spinal column.

It should be understood that the lobes of the various embodiments and corresponding bores of the introducer or introduction instrument 18 could have the same general dimension or size or they could have different sizes. Also, they could have axes that are parallel and lie in the same plane, are parallel and lie in different planes, or non-parallel and lie in either the same or different planes.

Advantageously, the system, implants, introducers or introduction instruments shown provides a system and process for quickly and easily preparing or creating an implant receiving area, such as the implant receiving area 28 (FIG. 13), so that a complementary-shaped multi-lobe implant, such as implant 12, may be inserted therein.

In the illustration being described, the introducer or introduction instrument 18 and the implant 12 are typically made of stainless steel, but it should be understood that it could be made of any suitable material that is capable of performing the functions described herein. Thus, for example, the introducer or introduction instrument 18 and the implant 12 could be made of a polymer material, plastic, composite material, metallic material, such as titanium, ceramic, carbon fiber or other suitable material.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. An implant comprising:
an implant housing having an outer housing wall;
said outer housing wall comprising a plurality of implant lobes.

2. The implant as recited in claim 1 wherein said plurality of implant lobes are at least partly cylindrical and extend generally parallel to a longitudinal axis of said implant housing.

3. The implant as recited in claim 2 wherein said implant comprises a joining portion for joining said plurality of implant lobes where said plurality of implant lobes intersect.

4. The implant as recited in claim 3 wherein said joining portion defines a channel.

5. The implant as recited in claim 2 wherein said outer housing wall generally defines an open-eight shape in cross section.

6. The implant as recited in claim 1 wherein said plurality of implant lobes are generally the same size in cross section.

7. The implant as recited in claim 1 wherein said plurality of implant lobes are different sizes in cross section.

8. The implant as recited in claim 1 wherein each of said plurality of implant lobes defines a partial circumference of less than 360 degrees.

9. The implant as recited in claim 8 wherein said plurality of implant lobes are generally the same size in cross section.

10. The implant as recited in claim 8 wherein said plurality of implant lobes are different sizes in cross section.

11. The implant as recited in claim 1 wherein said implant housing comprises an elongated channel generally arranged at an intersection between each adjacent pair of said plurality of lobes.

12. The implant as recited in claim 1 wherein said implant is a tri-lobate.

13. The implant as recited in claim 11 wherein said plurality of implant lobes comprise a first implant lobe defined by a first implant lobe wall, a second implant lobe defined by a second implant lobe wall, a third implant lobe defined by a third implant lobe wall and a fourth implant lobe defined by a fourth implant lobe wall;
said first and second implant lobe walls generally intersecting at a first intersection and said third and fourth implant lobe walls generally intersecting at a second intersection.

14. The implant as recited in claim 13 wherein said implant housing further comprises a first elongated channel associated with said first intersection and a second elongated channel associated with said second intersection.

15. The implant as recited in claim 13 wherein said first and second implant lobe walls are situated generally opposite and parallel to said third and fourth implant lobe walls.

16. The implant as recited in claim 1 wherein said plurality of implant lobes comprise a first implant lobe and a second implant lobe adapted and sized to be received in an implant guide having a plurality of complementary-shaped guide passageways or bores.

17. The implant as recited in claim 1 wherein said implant housing comprises at least one bone-graft area for receiving bone graft material.

18. The implant as recited in claim 5 wherein said implant comprises four or more lobes.

19. An implant-drill guide comprising:
a housing having a plurality of passage wall portions defining a plurality of passages or bores extending through said housing;
said plurality of passage wall portions intersecting and cooperating to define at least one implant guide passageway through said housing, said plurality of passages or bores being in communication and each being adapted to receive and guide at least one drill bit for drilling into bone in order to define or provide an implant receiving area for receiving an implant;
said at least one implant guide passageway being adapted to receive and guide said implant into said implant receiving area, said implant having a cross-sectional shape that generally complements a cross-sectional shape of said plurality of passages or bores.

20. The implant-drill guide as recited in claim 19 wherein said plurality of passage wall portions cooperate to define said implant passageway as an open-eight shape in cross section.

21. The implant-drill guide as recited in claim 19 wherein said plurality of passages or bores are generally parallel to a longitudinal axis of said housing.

22. The implant-drill guide as recited in claim 19 wherein said housing comprises at least one window that is in communication with said implant guide passageway.

23. The implant-drill guide as recited in claim 19 wherein each of said plurality of passage wall portions is, at least in part, generally cylindrical until said plurality of passages or bores intersect.

24. The implant-drill guide as recited in claim 19 wherein said plurality of passage wall portions are generally the same size in cross section.

25. The implant-drill guide as recited in claim 19 wherein each of said plurality of passage wall portions define or prescribe at least a partial circumference.

26. The implant-drill guide as recited in claim 25 wherein said at least a partial circumference of said plurality of passage wall portions are generally the same size.

27. The implant-drill guide as recited in claim 25 wherein said at least a partial circumference of said plurality of passage wall portions are generally different sizes.

28. The implant-drill guide as recited in claim 19 wherein said housing comprises at least one male guide projection, protuberance or prolongation.

29. The implant-drill guide as recited in claim 28 wherein said housing comprises a plurality of male guide projections, protuberances or prolongations, each having a height dimensional and being generally smaller than a cross-sectional height of said housing, with said plurality of male guide projections being adapted to be received in said implant receiving area between adjacent vertebrae.

30. The implant-drill guide as recited in claim 29 wherein said plurality of passages or bores are dimensioned and adapted to define a drill jig for guiding at least one drill bit through each of said plurality of passages or bores to drill a predetermined shape into at least one or both of said adjacent vertebrae.

31. The implant-drill guide as recited in claim 30 wherein said predetermined shape comprises a plurality of arcuate or curved bore sections, said implant comprising at least one implant lobe having a shape that generally complements a shape of said arcuate or curved bore sections.

32. The implant-drill guide as recited in claim 19 wherein said plurality of passages or bores comprise:
a first elongated passageway;
a second elongated passageway;
said first and second elongated passageways being in communication, intersecting and being at least partly cylindrical.

33. The implant-drill guide as recited in claim 32 wherein said first and second elongated passageways are the same size in cross section.

34. The implant-drill guide as recited in claim 32 wherein said first and second elongated passageways are different sizes in cross section.

35. The implant-drill guide as recited in claim 19 wherein said plurality of passage wall portions intersect and cooperate to define a predetermined passageway wall shape, said implant having an implant shape that generally complements said predetermined wall shape.

36. The implant-drill guide as recited in claim 35 wherein said predetermined passageway wall shape generally defines an open-eight shape.

37. The implant-drill guide as recited in claim 30 wherein said plurality of passage wall portions cooperate to define a predetermined passageway wall shape defining a plurality of arcuate or curved seats;
said implant having an implant shape that generally complements said predetermined passageway wall shape, said implant shape defining a plurality of lobes that become seated in said plurality of arcuate or curved seats.

38. The implant-drill guide as recited in claim 19 wherein a predetermined passageway wall shape generally defines an open-eight shape.

39. The implant-drill guide as recited in claim 19 wherein said housing comprises at least one window for viewing the implant and drill bit when they pass through at least one of said plurality of passages or bores.

40. The implant-drill guide as recited in claim 19 wherein said plurality of passages or bores define a bi-lobate or tri-lobate shape

41. An implant system comprising:
an implant-drill guide comprising an implant-drill housing having a plurality of passage wall portions defining a plurality of passages or bores extending through said implant-drill housing, said plurality of passages wall portions intersecting and cooperating to define at least one implant guide passageway through said implant-drill housing, said plurality of passages or bores being in communication and at least one of said plurality of passages being adapted to receive and guide at least one drill bit for drilling into bone in order to facilitate defining or providing an implant receiving area for receiving an implant, said implant receiving area having at least one drill lobe area created by said at least one drill bit;
said implant having an implant housing having an outer housing wall, said outer housing wall comprising at least one implant lobe that generally complements a shape of and mates with said at least one drill lobe area;
said at least one implant guide passageway being adapted to receive and guide said implant into said implant receiving area.

42. The implant system as recited in claim 41 wherein said plurality of passage wall portions cooperate to define said implant passageway as an open-eight shape in cross section.

43. The implant system as recited in claim 41 wherein said plurality of passages or bores are generally parallel to a longitudinal axis of said housing.

44. The implant system as recited in claim 41 wherein each of said plurality of passages or bores are adapted to receive and guide a drill bit having a dimension slightly smaller than a dimension of said plurality of passages or bores.

45. The implant system as recited in claim 41 wherein said implant-drill housing comprises at least one window that is in communication with said implant guide passageway.

46. The implant system as recited in claim 41 wherein each of said plurality of passage wall portions are, at least in part, generally cylindrical until said plurality of passages or bores intersect.

47. The implant system as recited in claim 41 wherein said plurality of passage wall portions each define at least a partial circumference that are generally the same size.

48. The implant system as recited in claim 47 wherein each of said plurality of passage wall portions define at least a partial circumference that are different sizes.

49. The implant system as recited in claim 47 wherein said plurality of passage wall portions each define at least a partial circumference that are not the same size.

50. The implant system as recited in claim 49 wherein at least a partial circumference of said plurality of passage wall portions are generally the same size.

51. The implant system as recited in claim 41 wherein said implant-drill housing comprises at least one male guide projection, protuberance or projection for aligning said implant-drill guide with a disc area between adjacent bones so that said at least one drill bit can drill at least one of said adjacent bone as said at least one drill bit is guided through said implant-drill housing.

52. The implant system as recited in claim 51 wherein said implant-drill housing comprises a plurality of male guide projections, protuberances or projections, each having a height that is generally smaller than a cross-sectional height of said implant drill housing, with said plurality of male guide projections being adapted to be received in said implant receiving area between adjacent vertebrae so that said plurality of passages or bores may guide both said drill bit and said implant into said implant.

53. The implant system as recited in claim 52 wherein said implant further comprises at least one drill bit, said plurality of passages or bores are dimensioned and adapted to define a drill jig for guiding said at least one drill bit through each of said plurality of passages or bores to drill a predetermined shape into at least one or both of said adjacent vertebrae.

54. The implant system as recited in claim 53 wherein said implant comprises a plurality of longitudinal lobes, said predetermined shape comprises a plurality of said drill lobe areas that define arcuate or curved bore sections, said implant comprising a plurality of implant lobes having a shape that generally complements a shape of said arcuate or curved bore sections.

55. The implant system as recited in claim 41 wherein said plurality of passages or bores comprise:
a first elongated passageway;
a second elongated passageway;
said first and second elongated passageways being in communication, intersecting and being at least partly cylindrical.

56. The implant system as recited in claim 55 wherein said first and second elongated passageways are adapted to define a drill jig for drilling a predetermined shape into at least one bone.

57. The implant system as recited in claim 56 wherein said implant comprises a plurality of implant lobes, said predetermined shape being adapted and dimensioned to capture said plurality of implant lobes such that a tension or compression is applied against said implant to facilitate keeping said implant in said implant receiving area after implantation.

58. The implant system as recited in claim 55 wherein said first and second elongated passageways are different sizes in cross section.

59. The implant system as recited in claim 55 wherein said first and second elongated passageways are generally the same size in cross section.

60. The implant system as recited in claim 41 wherein said plurality of passage wall portions cooperate to define a multi-lobe passageway shape defining a plurality of arcuate or curved seats in bone;
said implant having a plurality of lobes that generally complements said multi-lobe passageway shape and that become oriented or seated in said plurality of arcuate or curved seats in said implant-drill housing.

61. The implant system as recited in claim 60 wherein said multi-lobe passageway shape generally defines an open-eight shape.

62. The implant system as recited in claim 60 wherein each of said multi-lobe passageway shapes and said plurality of lobes are generally curved or arcuate.

63. The implant system as recited in claim 41 wherein said implant-drill housing comprises at least one window for viewing said implant and said at least one drill bit when they pass through at least one of said plurality of passages or bores.

64. The implant system as recited in claim 57 wherein said plurality of implant lobes are at least partly cylindrical and extend generally parallel to a longitudinal axis of said implant housing.

65. The implant system as recited in claim 64 wherein said outer housing generally defines an open-eight shape in cross section.

66. The implant system as recited in claim 41 wherein said at least one implant lobe is generally the same size in cross section.

67. The implant system as recited in claim 57 wherein said plurality of implant lobes are different sizes in cross section.

68. The implant system as recited in claim 41 wherein each of said plurality of passage wall portions defines a partial circumference of less than 360 degrees in cross section.

69. The implant system as recited in claim 57 wherein said plurality of implant lobes are generally the same size in cross section.

70. The implant system as recited in claim 57 wherein said implant housing comprises an elongated channel generally arranged at an intersection between each adjacent pair of said plurality of implant lobes.

71. The implant system as recited in claim 57 wherein said plurality of implant lobes comprise a first implant lobe defined by a first implant lobe wall, a second implant lobe defined by a second implant lobe wall, a third implant lobe defined by a third implant lobe wall and a fourth implant lobe defined by a fourth implant lobe wall;
said first and second implant lobe walls are situated generally opposite said third and fourth implant lobe walls.

72. The implant system as recited in claim 41 wherein said implant comprises a plurality of implant lobes comprising a first implant lobe and a second implant lobe adapted and sized to be received in said plurality of passages or bores.

73. The implant system as recited in claim 41 wherein said implant housing comprises at least one bone-graft area for receiving bone graft material.

74. The implant system as recited in claim 41 wherein said implant system is for lateral insertion of said implant.

75. The implant as recited in claim 1 wherein said plurality of implant lobes have axes that lie in the same plane.

76. The implant as recited in claim 1 wherein said plurality of implant lobes have axes that lie in different planes.

77. The implant as recited in claim 1 wherein said plurality of implant lobes are different sizes.
